(19) United States
(12) Patent Application Publication
(10) Pub. No.: US 2022/0265831 A1
(43) Pub. Date: Aug. 25, 2022

(54) Alginate Based Particles as a Temporary Embolic Agent

(71) Applicant: Crannmed Limited, Co. Galway (IE)

(72) Inventor: Sankalp Agarwal, Jaripatka, Nagpur (IN)

(73) Assignee: Crannmed Limited, Co. Galway (IE)

(21) Appl. No.: 17/636,811

(22) PCT Filed: Aug. 27, 2020
(86) PCT No.: PCT/IB2020/058022

§ 371 Date: Feb. 18, 2022

Related U.S. Application Data
(60) Provisional application No. 62/892,097, filed on Aug. 27, 2019.

(51) Int. Cl. A61K 47/36; A61M 5/19; A61M 5/28; A61L 24/00

(52) U.S. Cl. CPC A61K 47/36; A61M 5/19; A61M 5/286; A61L 24/0042; A61L 2300/604; A61L 2300/62; A61L 2430/36

(57) ABSTRACT

The present disclosure provides compositions including self-degrading alginate particles comprising alginate, alginate lyase, and divalent metal ions. The present disclosure also provides methods of making compositions including self-degrading alginate particles comprising alginate, alginate lyase, and divalent metal ions. The present disclosure also provides methods of inducing an embolism in a subject in thereof, and syringes containing the compositions of the present disclosure for use in the methods thereof.

### CROSS-REFERENCE TO RELATED APPLICATIONS

This application claims the benefit of U.S. Provisional Application No. 62/892,097 filed on Aug. 27, 2019, which is hereby incorporated by reference in its entirety for all purposes.

### BACKGROUND

Artificial blocking of a blood vessel, or embolization, in an organ may be used, for example, (a) to control bleeding caused due to trauma, (b) to prevent blood flow into abnormal blood vessels such as aneurysms, and/or (c) to treat an organ (e.g., to excise a tumor, for transplant, or for surgery). In many circumstances, the permanent embolization of blood vessels is not required. For such medical interventions, using temporary and bioresorbable embolic agents are desirable. For example, IMP/CS (Imipenem/Ciliastatin) antibiotic particles of size ranging from 10 μm to 80 μm have been used as a temporary embolic agent, however this material can require nearly a month to become absorbed completely (see, e.g., Okuno, et al.; “Midterm Clinical Outcomes and MR Imaging Changes after Transcatheter Arterial Embolization as a Treatment for Mild to Moderate Radiographic Knee Osteoarthritis Resistant to Conservative Treatment”, J. Vasc. Interv. Radiol. 2017; 28:995-1002). Similarly, other embolic agents such as Gelfoam®, collagen, and thrombin have also been used (see, e.g., Vaidya, et al.; “An overview of embolic agents”, Semin. Intervent. Radiol. 2008; 25:204-15). However, existing agents have numerous drawbacks such as unpredictable dissolution rate, lack of agent(s) that selectively degrade abovementioned matrices, and/or migration of the embolic agents causing non-specific occlusion (see, e.g., U.S. Patent Application Publication No. 20130211249). Furthermore, some embolic agents require a processing or preparation step before their use within the body. For example, Gelfoam has to be cut up into pledgets or slurried.

Accordingly, there is a need for embolic agents that can selectively degrade abovementioned matrices, and/or exhibit predictable dissolution rate without creating any non-specific occlusion in vivo.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BRIEF SUMMARY

In some aspects, the present disclosure provides a self-degrading alginate particle. In some embodiments, the self-degrading alginate particle comprises alginate molecules. In some embodiments, the alginate molecules have one or both of (i) a predetermined molecular weight, and (ii) a predetermined ratio of β-D-Mannuronic acid (M) blocks to α-L-Guluronic acid (G) blocks. In some embodiments, the self-degrading alginate particle comprises alginate lyase enzymes. In some embodiments, the self-degrading alginate particle comprises metal ions. In some embodiments the metal ions cross-link the alginate molecules. In some embodiments the metal ions cross-link the alginate molecules to form an alginate matrix.

In some embodiments, a degradation of the alginate particle in vivo or in vitro is controlled by one or more of the predetermined molecular weight of the alginate molecules, the predetermined ratio of M to G blocks, a concentration of the alginate lyase enzyme, a concentration of the metal ions, and a binding affinity of the metal ions. In some embodiments, a degradation of the alginate particle in vivo or in vitro is controlled by the predetermined molecular weight of the alginate molecules. In some embodiments, the predetermined molecular weight is greater than about 100 kilodaltons (kD). In some embodiments, the predetermined molecular weight is greater than about 200 kilodaltons (kD). In some embodiments, the predetermined molecular weight is greater than about 800 kilodaltons (kD).

In some embodiments, a degradation of the alginate particle in vivo or in vitro is controlled by the predetermined ratio of M to G blocks. In some embodiments, the predetermined ratio of M to G blocks is about 50:50, about 55:45, about 60:40, about 65:35, about 70:30, about 75:25, about 80:20, about 85:15, about 90:10, or about 95:5. In some embodiments, the alginate particle degrades over a period of less than about 5 days. In some embodiments, the alginate particle degrades over a period of greater than about 2 days. In some embodiments, the predetermined ratio of M to G blocks is about 50:50, about 45:55, about 40:60, about 35:65, about 30:70, about 25:75, about 20:80, about 15:85, about 10:90, or about 5:95. In some embodiments, the alginate particle degrades over a period of between about 5 days and about 30 days. In some embodiments, a degradation of the alginate particle is controlled by a concentration of the alginate lyase enzyme.

In some embodiments, the activity of the alginate lyase enzyme is between about 0.05 mU (milliunits) and about 2.5 mU per particle. In some embodiments, the alginate particle degrades over a period of less than about 5 days. In some embodiments, the alginate lyase enzyme is between about 0.05 nU (nanounits) and about 0.05 mU per particle. In some embodiments, the alginate particle degrades over a period of between about 5 days and about 30 days. In some embodiments, the activity of the alginate lyase enzyme is less than about 0.05 nU per particle. In some embodiments, the alginate particle degrades over a period of greater than about 30 days.

In some embodiments, a degradation of the alginate particle is controlled by a binding affinity of the metal ions. In some embodiments, the metal ion is a cation. In some embodiments, the cation is selected from the group consisting of Cu²⁺, Ba²⁺, Sr²⁺, Ca²⁺, Co²⁺, Ni²⁺, Mn²⁺, and Mg²⁺. In some embodiments, the cation is Ba²⁺. In some embodiments, the cation is Ca²⁺.

In some embodiments, a diameter of the alginate particle is between about 40 microns (μm) and about 2000 μm. In some embodiments, the diameter of the alginate particle is between about 40 μm and about 1000 μm. In some embodiments, the diameter of the alginate particle is between about 40 μm and about 200 μm. In some embodiments, the self-degrading alginate particles further comprises one or more alginate lyase inhibitors. In some embodiments, the one or more alginate lyase inhibitors are independently selected from the group consisting of Cu²⁺, Zn²⁺, Fe³⁺, Ca²⁺ and Mg²⁺. In some embodiments, the self-degrading alginate particles further comprises a cryoprotectant. In some embodiments, the cryoprotectant is selected from the group consisting of sucrose, glycerol, ethylene glycol, sorbitol, trehalose, and propylene glycol.

In some embodiments, a sphericity of the alginate particle is at least about 0.7, at least about 0.75, at least about 0.8, at least about 0.85, at least about 0.9, at least about 0.95, or at least about 0.99. In some embodiments, the alginate molecules comprise oxidized alginate molecules. In some embodiments, the self-degrading alginate particles further comprises a therapeutically effective amount of an active ingredient. In some embodiments, the metal ions comprise divalent metal ions or trivalent metal ions. In some embodiments, the alginate lyase enzymes are entrapped by the cross-linked alginate molecules.

In some aspects, the present disclosure provides a method of inducing a self-degrading embolism in a subject in need thereof. In some embodiments, the method comprises administering a plurality of the alginate particles of the present disclosure into a blood vessel of the subject. In some embodiments, the blood vessel is a geniculate artery.

In some aspects, the present disclosure provides a syringe. In some embodiments, the syringe comprises a first chamber. In some embodiments, the first chamber contains dried alginate particles of present disclosure. In some embodiments, the syringe comprises a second chamber. In some embodiments, the second chamber is disposed axially to the first chamber. In some embodiments, the second chamber contains a reconstitution medium. In some embodiments, the syringe comprises a plunger. In some embodiments, the plunger is configured to, upon depression, expose the dried alginate particles to the reconstitution medium, thereby reconstituting the dried alginate particles.

In some embodiments, the syringe further comprises a breakable membrane separating the first chamber and the second chamber, wherein upon depression of the plunger, the breakable membrane breaks to expose the dried alginate particles to the reconstitution medium, thereby reconstituting the dried alginate particles. In some embodiments, a degradation of the alginate particle in vivo or in vitro is controlled by one or more of the predetermined molecular weight of the alginate molecules, the predetermined ratio of M to G blocks, a concentration of the alginate lyase enzyme, a concentration of the metal ions, and a binding affinity of the metal ions.

In some embodiments, a degradation of the alginate particle in vivo or in vitro is controlled by the predetermined molecular weight of the alginate molecules. In some embodiments, the predetermined molecular weight is greater than about 100 kilodaltons (kD). In some embodiments, the predetermined molecular weight is greater than about 200 kilodaltons (kD). In some embodiments, the predetermined molecular weight is greater than about 800 kilodaltons (kD). In some embodiments, a degradation of the alginate particle in vivo or in vitro is controlled by the predetermined ratio of M to G blocks. In some embodiments, the predetermined ratio of M to G blocks is about 50:50, about 55:45, about 60:40, about 65:35, about 70:30, about 75:25, about 80:20, about 85:15, about 90:10, or about 95:5. In some embodiments, the alginate particle degrades over a period of less than about 5 days. In some embodiments, the alginate particle degrades over a period of greater than about 2 days. In some embodiments, the predetermined ratio of M to G blocks is about 50:50, about 45:55, about 40:60, about 35:65, about 30:70, about 25:75, about 20:80, about 15:85, about 10:90, or about 5:95. In some embodiments, the alginate particle degrades over a period of between about 5 days and about 30 days. In some embodiments, a degradation of the alginate particle is controlled by a concentration of the alginate lyase enzyme.

In some embodiments, the activity of the alginate lyase enzyme is between about 0.05 mU and about 2.5 mU per particle. In some embodiments, the alginate particle degrades over a period of less than about 5 days. In some embodiments, the activity of the alginate lyase enzyme is between about 0.05 nU and 0.05 mU per particle. In some embodiments, the alginate particle degrades over a period of between about 5 days and about 30 days. In some embodiments, the activity of the alginate lyase enzyme is less than about 0.05 nU per particle. In some embodiments, the alginate particle degrades over a period of greater than about 30 days.

In some embodiments, a degradation of the alginate particle is controlled by a binding affinity of the metal ions. In some embodiments, the metal ion is a cation. In some embodiments, the cation is selected from the group consisting of Cu²⁺, Ba²⁺, Sr²⁺, ca²⁺, Co²⁺, Ni²⁺, Mn²⁺, and Mg²⁺. In some embodiments, the cation is Ba²⁺. In some embodiments, the cation is Ca²⁺. In some embodiments, a diameter of the alginate particle is between about 40 microns (μm) and about 2000 μm. In some embodiments, the diameter of the alginate particle is between about 40 μm and about 1000 μm. In some embodiments, the diameter of the alginate particle is between about 40 μm and about 200 μm. In some embodiments, the dried alginate particles further comprise one or more alginate lyase inhibitors independently selected from the group consisting of Cu²⁺, Zn²⁺, Fe³⁺, Ca²⁺ and Mg²⁺. In some embodiments, the dried alginate microspheres further comprise a cryoprotectant. In some embodiments, the cryoprotectant is selected from the group consisting of sucrose, glycerol, ethylene glycol, sorbitol, trehalose, and propylene glycol.

In some embodiments, a sphericity of the alginate particle is at least about 0.7, at least about 0.75, at least about 0.8, at least about 0.85, at least about 0.9, at least about 0.95, or at least about 0.99. In some embodiments, the alginate molecules comprise oxidized alginate molecules. In some embodiments, the dried alginate particles further comprise a therapeutically effective amount of an active ingredient. In some embodiments, the metal ions comprise divalent metal ions or trivalent metal ions. In some embodiments, the dried alginate particles comprise alginate lyase enzymes entrapped by cross-linked alginate molecules.

In some aspects, the present disclosure provides a method of preparing a self-degrading alginate particle. In some embodiments, the method comprises obtaining a first composition comprising alginate microspheres. In some embodiments, the alginate microspheres comprise alginate molecules having one or both of (i) a predetermined molecular weight, and (ii) a predetermined ratio of β-D-Mannuronic acid (M) blocks to α-L-Guluronic acid (G) blocks. In some embodiments, the method comprises mixing the first composition with a second composition. In some embodiments, the second composition comprises alginate lyase enzymes and metal ions, thereby creating a mixture. In some embodiments, the method comprises preparing a self-degrading alginate particle from the mixture.

In some embodiments, the method further comprises inhibiting degradation of the alginate molecules in the mixture by one or both of: (i) maintaining a pH of the mixture at less than about 6.5; and (ii) maintaining a temperate of the mixture at less than about 10 degrees Celsius (° C.). In some embodiments, the pH of the mixture is maintained at between about 3 and about 6.5. In some embodiments, the temperature of the mixture is maintained at between about 4° C. and about 10° C. In some embodiments, the preparing comprises performing a water-in-oil emulsion technique or a droplet technique. In some embodiments, the method further comprises reconstituting the self-degrading alginate particle in a solution having a pH of between about 6.8 and about 7.5.

In some embodiments, a degradation of the alginate particle in vivo or in vitro is controlled by one or more of the predetermined molecular weight of the alginate molecules, the predetermined ratio of M to G blocks, a concentration of the alginate lyase enzyme, a concentration of the metal ions, and a binding affinity of the metal ions. In some embodiments, a degradation of the alginate particle in vivo or in vitro is controlled by the predetermined molecular weight of the alginate molecules. In some embodiments, the predetermined molecular weight is greater than about 100 kilodaltons (kD). In some embodiments, the predetermined molecular weight is greater than about 200 kilodaltons (kD). In some embodiments, the predetermined molecular weight is greater than about 800 kilodaltons (kD). In some embodiments, a degradation of the alginate particle in vivo or in vitro is controlled by the predetermined ratio of M to G blocks. In some embodiments, the predetermined ratio of M to G blocks is about 50:50, about 55:45, about 60:40, about 65:35, about 70:30, about 75:25, about 80:20, about 85:15, about 90:10, or about 95:5. In some embodiments, the alginate particle degrades over a period of less than about 5 days. In some embodiments, the alginate particle degrades over a period of greater than about 2 days. In some embodiments, the predetermined ratio of M to G blocks is about 50:50, about 45:55, about 40:60, about 35:65, about 30:70, about 25:75, about 20:80, about 15:85, about 10:90, or about 5:95.

In some embodiments, the alginate particle degrades over a period of between about 5 days and about 30 days. In some embodiments, a degradation of the alginate particle is controlled by a concentration of the alginate lyase enzyme. In some embodiments, the activity of the alginate lyase enzyme is between 0.05 mU and 2.5 mU per particle. In some embodiments, the alginate particle degrades over a period of less than about 5 days. In some embodiments, the concentration of the alginate lyase enzyme is between about 0.05 nU to 0.05 mU per particle. In some embodiments, the alginate particle degrades over a period of between about 5 days and about 30 days. In some embodiments, the activity of the alginate lyase enzyme is less than about 0.05 nU per particle. In some embodiments, the alginate particle degrades over a period of greater than about 30 days. In some embodiments, a degradation of the alginate particle is controlled by a binding affinity of the metal ions. In some embodiments, the metal ion is a cation. In some embodiments, the cation is selected from the group consisting of Cu²⁺, Ba²⁺, Sr²⁺, Ca²⁺, Co²⁺, Ni²⁺, Mn²⁺, and Mg²⁺. In some embodiments, the cation is Ba²⁺.

In some embodiments, the cation is Ca²⁺. In some embodiments, a diameter of the alginate particle is between about 100 microns (μm) and about 2000 μm. In some embodiments, the diameter of the alginate particle is between about 100 μm and about 1000 μm. In some embodiments, the diameter of the alginate particle is between about 100 μm and about 200 μm.

In some embodiments, the alginate particle further comprises one or more alginate lyase inhibitors independently selected from the group consisting of Cu²⁺, Zn²⁺, Fe³⁺, Ca²⁺ and Mg²⁺.

In some embodiments, the alginate particle further comprises a cryoprotectant. In some embodiments, the method further comprises adding the cryoprotectant to the alginate particle prior to lyophilizing the alginate particle. In some embodiments, the cryoprotectant is selected from the group consisting of sucrose, glycerol, ethylene glycol, sorbitol, trehalose, and propylene glycol.

In some embodiments, a sphericity of the alginate particle is at least about 0.7, at least about 0.75, at least about 0.8, at least about 0.85, at least about 0.9, at least about 0.95, or at least about 0.99. In some embodiments, the alginate molecules comprise oxidized alginate molecules. In some embodiments, the self-degrading alginate particle comprises a therapeutically effective amount of an active ingredient. In some embodiments, the metal ions comprise divalent metal ions or trivalent metal ions. In some embodiments, subsequent to mixing the first composition with the second composition, the metal ions cross-link the alginate molecules, and the alginate lyase enzymes are entrapped by the cross-linked alginate molecules.

In some aspects, the present disclosure provides a method of treating a subject in need thereof by temporarily embolizing a blood vessel. In some embodiments, the method comprises administering a plurality of the alginate particles of the present disclosure into the blood vessel of the subject. In some embodiments, the blood vessel in a geniculate artery. In some embodiments, the subject has a condition selected from the group consisting of knee pain, arthritis, shoulder pain from adhesive capsulitis, kidney lesions, liver lesions, uterine fibroids, benign prostate hyperplasia, arteriovenous malformations, nasopharyngeal angifibroma, cerebral aneurysm, gastrointestinal bleeding, variocele, surgical bleeding, splenic rupture, splenomegaly, obesity, and solid tumors. In some embodiments,

### BRIEF DESCRIPTION OF THE SEVERAL VIEWS OF THE DRAWINGS

The foregoing summary, as well as the following detailed description of embodiments of the compositions and fluid delivery devices, will be better understood when read in conjunction with the appended drawings of exemplary embodiments. It should be understood, however, that embodiments of the present disclosure are not limited to the precise arrangements and instrumentalities shown.

FIG. 1A illustrates the preparation and tailoring of properties of exemplary microspheres of the present disclosure. Alginate and alginate lyase dissolved in aqueous media. Microspheres prepared by conventional methods to gel alginate+lyase droplets by cationic crosslinking;

FIG. 1B illustrates the preparation and tailoring of properties of exemplary microspheres of the present disclosure. Microspheres are lyophilized with optional lyoprotectant to remove water and “freeze” enzyme activity, preventing premature degradation during storage. Microspheres are sterilized in this form;

FIG. 1C illustrates the preparation and tailoring of properties of exemplary microspheres of the present disclosure. Degradation properties may be controlled by varying lyase and alginate parameters and preparation conditions to produce particles of varied degradation rates from days to months depending upon indication to be treated;

FIG. 2A illustrates exemplary post-particle preparation processes and methods of use. Lyophilised alginate particles are prepared to remove water and freeze enzyme activity. Cyoprotectant protects enzyme and microsphere structure to allow shape recovery upon hydration;

FIG. 2B illustrates exemplary post-particle preparation processes and methods of use. Particles are reconstituted in aqueous media at point of use, hydrating the particle and enabling catalytic activity of the lyase;

FIG. 2C illustrates exemplary post-particle preparation processes and methods of use. Particles are prepared in an appropriate suspension for intra-arterial delivery for the designated embolization procedure (e.g., uterine fibroid embolization). When in the body, the enzyme activity is enhanced and the alginate chains are cleaved, releasing the cations, polymer chain fragments and lyase into the body where they may be resorbed or excreted;

FIG. 3A illustrates enzyme concentration dependent alginate particle degradation. A line graph of the degradation of alginate particles over time with varying enzyme concentrations is provided;

FIG. 3B illustrates enzyme concentration dependent alginate particle degradation. Images of the particles after the degradation period, and samples having varying concentrations of enzyme;

FIG. 4 illustrates in vitro biocompatibility of alginate lyase loaded-calcium ion alginate particles;

FIG. 5A illustrates ex vivo degradation studies of alginate lyase loaded calcium ion-complexed alginate particles in a liver model at 0 hours;

FIG. 5B illustrates ex vivo degradation studies of alginate lyase loaded calcium ion-complexed alginate particles in a liver model at 24 hours;

FIG. 5C illustrates ex vivo degradation studies of alginate lyase loaded calcium ion-complexed alginate particles in a liver model at 48 hours;

FIG. 6 illustrates pH dependent regulation of enzyme conformation/activity; and

FIG. 7 illustrates an exemplary syringe showing the compartments for the suspension medium and dried alginate microspheres. The separating membrane may be torn inside the syringe by applying pressure on the plunger, thereby reconstituting the dried alginate microsphere in the suspension medium containing calcium chloride solution.

### DETAILED DESCRIPTION

Overview

Generally, the present disclosure provides a method for the preparation of divalent metal ion complexed-alginate particles containing alginate lyase enzyme to control its degradation for use in embolization applications. In certain embodiments, the present disclosure relates to the field of polymer chemistry, biochemistry, immunology and particularly to the field of compositions for use in minimally invasive endovascular and non-vascular therapeutics.

Alginate-based liquid embolic agents have been considered as a promising tool for embolization. Pure forms of alginate are highly biocompatible, and their gelling properties may be controlled. They are naturally-occurring polysaccharide copolymers composed of randomly 1-4 linked β-D-mannuronic acid (M-block)-α-L-guluronic (G block) of various M: G ratios that are commonly found in various seaweeds. Alginate is dissolved in the contrast agent iohexol (e.g., to impart radiopacity) and is gelled into a hydrocoil form which hardens in the presence of calcium chloride solution due to ionic crosslinking of the carboxylate groups of the polysaccharide residues with Ca²⁺. All of these components are mixed simultaneously at the treatment site to create an in situ mass of gel (e.g., EmboGel). This gel may be subsequently dissolved (e.g., using a mixture such as EmboClear, which is a mixture of alginate lyase enzyme and ethylenediaminetetraacetic acid (EDTA)). The enzyme cleaves the polysaccharide chains at the glycosidic bond via a 0-elimination mechanism and the EDTA de-complexes the ionic cross-links by scavenging the Ca²⁺ by chelation. This dissolution agent is administered at the site of the embolus to clear the occluded vessel within a few minutes. However, these existing methods have several drawbacks as described below.

Firstly, the procedure to degrade the EmboGel using EmboClear solution introduces additional risk to the patient, as they must undertake additional post embolization procedures. Moreover, depending upon the time interval desired between the formation of the embolus and its dissolution, the patient may require a second visit, thereby incurring the associated costs for a re-catheterization procedure. Secondly, in some cases such as aneurysm therapy, the alginate gel could migrate to the parent artery during injection or after the post-embolization procedure which may cause non-specific vessel occlusion (see, e.g., Barnett, et al., “A selectively dissolvable radiopaque hydrogel for embolic applications”; and U.S. Pat. No. 9,220,761). In the latter case, non-specific migration of degraded/disintegrated alginate gels to other parts of the body predominantly occurs due to instant/uncontrolled degradation/disintegration of EmboGel by the EmboClear, causing generation of particulates of various size and that are unable to be reabsorbed before they are distributed to off-target distal locations at which the EmboClear becomes ineffective due to dilution. If EmboGel is loaded with a bioactive agent/drug, separate administration of EmboClear dissolution agent may be required in order to afford degradation-controlled release kinetics.

Furthermore, Kunjukunju, et al. reported alginate lyase aggregates of various size (10-300 μm) and shape using ammonium sulfate (see, e.g., Kunjukunju, et al., “Cross-linked enzyme aggregates of alginate lyase: A systematic engineered approach to controlled degradation of alginate hydrogel.” International Journal of Biological Macromolecules 115 (2018): 176-184). These aggregates were cross-linked using glutaraldehyde to produce insoluble catalytically active alginate lyase aggregates. The resultant cross-linked aggregate was encapsulated in an alginate hydrogel to achieve its controlled degradation. However, the method described in this report may not be suitable to enable the preparation of a temporary alginate-based embolization agent per se.

Firstly, it would not be possible to produce alginate particles of the desired size, as the size and polydispersity of the described aggregates of the enzyme could not be encapsulated. Secondly, the process outlined described crosslinking the enzyme aggregates with glutaraldehyde which is a toxic agent that should be avoided in the preparation of compositions intended for use in the human body. Thirdly, the authors did not report any other methods to control the degradation of the alginate, such as molecular weight or viscosity of sodium alginate, pre-treatment of the enzyme using modifiers (metal ions) or other physiochemical parameters such as pH and temperature or to improve the encapsulation efficiency of alginate lyase enzyme. Lastly, no work has been performed to achieve the storage and shelf life of the alginate aggregates.

In certain embodiments, the present disclosure proposes the in situ-controlled degradation of alginate lyase enzyme loaded alginate particles for embolic applications. Because the enzyme may be uniformly distributed in the alginate particles, this strategy gives one or more the following advantages over the existing temporary embolic agents and prior-art alginate-based systems.

The catalytic activity of alginate lyase enzyme may be controlled using modifiers (stimulatory or inhibitory). Additionally, the amount of enzyme loaded in the alginate particles may be used to control the degradation of the particles ranging from a few hours to weeks. This strategy may provide a predictable degradation rate of alginate particles which may be of prime importance for certain applications of embolic therapy. Such controlled degradation of the embolic agents has not been observed for existing temporary embolic agents (e.g., EmboGel).

Due to the controlled degradation of the matrix, the by-products or particulates may be reabsorbed and excreted through the kidneys. Therefore, the risk of non-specific occlusion of blood vessels is minimal.

As the alginate lyase enzyme loaded alginate particles undergo in situ degradation, the clinicians do not require post-embolization procedures for the dissolution of embolic particles and the patient is not exposed to additional procedure-related risk.

In the prior art-alginate based system, the composition of liquid dissolution agent comprises a large amount of alginate lyase enzyme to dissolve the divalent metal ions cross-linked alginate gel instantly. On the contrary, in certain embodiments, the present disclosure proposes the controlled degradation of the divalent metal ion-complexed alginate particles by loading the alginate lyase enzyme into the particles. The loading of alginate lyase enzyme into the particles may improve the recyclability efficiency of the enzyme. This reduces the amount of enzyme required for the degradation of the alginate when compared to the prior art-alginate based embolic agents.

These biodegradable embolic particles may also be loaded with drugs for delivery at the target site that may be controlled by the rate of degradation of the alginate matrix.

### Definitions

As used herein, the term “a”, “an”, or “the” generally is construed to cover both the singular and the plural forms.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” may refer to a range of ±20%, ±10%, or ±5% of a given numeric value.

The term “substantially” as used herein may refer to a majority of, or mostly, as in at least about 50%, 60%, 70%, 80%, 90%, 95%, 96%, 97%, 98%, 99%, 99.5%, 99.9%, 99.99%, or at least about 99.999% or more.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, or the like, which is nontoxic and which does not interact with other components of the composition in a deleterious manner.

The term “therapeutically effective amount” may generally refer to the amount (or dose) of a compound or other therapy that is minimally sufficient to prevent, reduce, treat or eliminate a condition, or risk thereof, when administered to a subject in need of such compound or other therapy. In some instances, the term “therapeutically effective amount” may refer to that amount of compound or other therapy that is sufficient to have a prophylactic effect when administered to a subject. The therapeutically effective amount may vary; for example, it may vary depending upon the subject's condition, the weight and age of the subject, the severity of the disease condition, the manner of administration (e.g., subcutaneous delivery) and the like, all of which may be determined by one of ordinary skill in the art.

As used herein, “treating” or “treat” includes: (i) preventing a pathologic condition from occurring (e.g., prophylaxis); (ii) inhibiting the pathologic condition or arresting its development; (iii) relieving the pathologic condition; and/or (iv) diminishing symptoms associated with the pathologic condition.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions of the disclosure is contemplated. Supplementary active ingredients may also be incorporated into the compositions.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

Compositions and Methods of Use

The present disclosure relates to the loading of the alginate lyase in the sodium alginate together gelled in the presence of divalent metal ions to form biodegradable alginate lyase loaded alginate particles. In some embodiments, the present disclosure provides an alginate particle. In certain embodiments, the present disclosure provides an alginate particle capable of controlled, self-degradation. In certain embodiments, the present disclosure provides a self-degrading alginate particle with controlled degradation properties.

The amount of enzyme loaded in the alginate particles and pre-treatment of the enzyme using modifiers are used to modulate the degradation rate of alginate particles. This involves the mixing of native or modified alginate lyase with sodium alginate in different proportions. This is followed by the pre-treatment of the alginate lyase-sodium alginate solution to prevent its degradation during the manufacturing of the particles. These particles are prepared by creating uniform droplets of alginate lyase-sodium alginate solution gelled in a divalent metal ions bath which optionally contains one or more of cryoprotectant to protect the composition during lyophilization of the particles. The chemical properties of alginates change with the molecular weight and the ratio of M (β-D-mannuronic acid) and G (α-L-guluronic acid) blocks (M/G) (Ramos, et al., “Effect of alginate molecular weight and M/G ratio in beads properties foreseeing the protection of probiotics”, Food Hydrocoll. 2018; 77:8-16). Particularly, the G-block has more affinity toward divalent cations as compared to the M-block due to the geometry of the carboxylate residues. Alginate contains a large variation in the M and G content, and also possesses the variation in the sequence structures (G-block, M-block and MG block). In general, the alginate with a higher G content relative to M content (lower M/G ratio) when cross-linked with cations gives mechanically robust structures/capsules with low permeability.

On the other hand, alginates with a lower G content relative to M content (higher M/G ratio) gives weaker strength gel having a high permeability matrix. Ramos, et al. reported that low M/G ratio and lower molecular weight alginate produced less permeable and stronger alginate beads cross-linked with calcium ions. Other factors which improve the robustness of alginate are choice of divalent ions and molecular weight/viscosity of alginates. Usually, for an endovascular procedure, the alginate particles mixed into a homogeneous suspension using two syringes connected by and three-way stop-cock and are delivered inside the body through a microcatheter, during which they may experience mechanical forces. Therefore, the mechanical robustness of the particles should be sufficient to maintain their integrity during routine use.

To achieve the rapid degradation (>=2 days to <=5 days) of alginate lyase-loaded di-valent metal ion complexed alginate particles, lower G-content alginate (e.g., higher M: G ratio) having low molecular weight/low viscosity may be used. In certain embodiments, the purified alginate contains more than 50% M content (β-D-mannuronic acid). The percentage of M-content in the purified alginate maybe 50% and 80%, 55%-75% and 60%-80%. To get intermediate (>5 days to <=30 days) or slow (>30 days) degradation periods the higher G content alginate (e.g., the lower M:G ratio) having high molecular weight/viscosity may be used. In certain embodiments, the purified alginate contains more than 50% G content (α-L-guluronic acid). The percentage of G-content in the purified alginate may be 50% and 80%, 55%-75% and 60%-80%.

The molecular weight or viscosity of alginate also affect the mechanical properties of the alginate particle (Farrés, et al., “Formation kinetics and rheology of alginate fluid gels produced by in-situ calcium release”, Food Hydrocolloids 40 (2014): 76-84). The average molecular weight of alginate polymers may be >100 kD, preferably >200 kD and most preferably >30 kD. The viscosity of 1% alginate solution at 20° C. may have a range >25 mPa-s, preferably <1000 mPa-s for the preparation of rapid and slow degrading alginate lyase loaded di-valent alginate particles.

The concentration of purified alginate or oxidized form of alginate can also affect the pore size and robustness of the divalent complexed alginate bead. The concentration of the alginate can be about 0.05% weight by volume (w/v), 0.10% w/v, 0.15% w/v, 0.20% w/v, 0.25% w/v, 0.30% w/v, 0.35% w/v, 0.40% w/v, 0.45% w/v, 0.50% w/v, 0.60% w/v, 0.70% w/v, 0.80% w/v, 0.90% w/v, 1.0% w/v, 1.25% w/v, 1.5% w/v, 1.75% w/v, 2.0% w/v, 2.25% w/v, 2.5% w/v, 2.75% w/v, 3.0% w/v, 3.25% w/v, 3.5% w/v, 3.75% w/v, 4% w/v, 4.25% w/v, 4.5% w/v, 4.75% w/v, 5.0% w/v, 5.25% w/v, 5.5% w/v, 5.75% w/v, 6.0% w/v, or greater than about 6.0% w/v for the preparation of rapid (>=2 days to <=5 days) and slow degrading (>5 days to <=30 days) alginate lyase loaded di-valent alginate particles.

In addition, the gelling time during which the alginate particles are crosslinked within the metal ion bath can also affect the size, sphericity and physical robustness of the divalent metal ion complexed alginate particles. Generally, the term “sphericity” can refer to a measure of how closely the shape of an object resembles that of a perfect sphere. The roundness of an injectable substance can be important, for example, as abnormally shaped substances can have difficulty in travelling through blood vessels, leading to clogged blood vessels, thereby blocking blood flow to various parts of the body The gelling time can be less than about 1 min, less than about 2 minutes, less than about 3 minutes, less than about 4 minutes, less than about 5 minutes, less than about 6 minutes, less than about 7 minutes less than about 8 minutes, less than about 9 minutes, less than about 10 minutes, less than about 11 minutes, less than about 12 minutes, less than about 13 minutes, less than about 14 minutes, less than about 15 minutes, less than about 20 minutes, less than about 25 minutes, or less than about 30 minutes.

To achieve the desired degradation period of the alginate lyase loaded alginate particles, the amount of enzyme mixed with preferred alginate may be varied. The amount of alginate lyase mixed with the alginate varies from <1 unit to 50 units/ml of sodium alginate for the preparation of rapid (>=2 days to <=5 days) intermediate (>5 days to <=30 days), or slow (>30 days) degrading di-valent complexed alginate particles. For the sake of clarity, in enzymology 1 unit (U) is the amount of enzyme that catalyses the reaction of 1 μmol of substrate per minute. The amount of enzyme loading into the di-valent complexed alginate particles also depends on the molecular weight or viscosity of the sodium alginate.

In certain embodiments, the activity of the alginate lyase enzyme is about 0.001 nanounits (nU) per particle, about 0.01 nU per particle, about 0.10 nU per particle, about 0.50 nU per particle, about 0.001 milliunits (mU) per particle, about 0.01 mU per particle, about 0.05 mU per particle, about 0.10 mU per particle, about 0.25 mU per particle, about 0.50 mU per particle, about 0.75 mU per particle, about 1.0 mU per particle, about 1.25 mU per particle, about 1.5 mU per particle, about 1.75 mU per particle, about 2.0 mU per particle, about 2.25 mU per particle, about 2.5 mU per particle, about 2.75 mU per particle, about 3.0 mU per particle, about 3.25 mU per particle, about 3.5 mU per particle, about 3.75 mU per particle, about 4.0 mU per particle, or a range between any two values thereof. In certain embodiments, the activity of the alginate lyase enzyme is between about 0.001 mU and 4.0 mU per particle. In certain embodiments, the activity of the alginate lyase enzyme is between about 0.01 mU and 3 mU per particle. In certain embodiments, the activity of the alginate lyase enzyme is between about 0.05 mU and 2.5 mU per particle. In certain embodiments, the activity of the alginate lyase enzyme is between about 0.05 mU and 0.5 mU per particle. In certain embodiments, the activity of the alginate lyase enzyme is between about 0.5 mU and 1.0 mU per particle. In certain embodiments, the activity of the alginate lyase enzyme is between about 1.0 mU and 1.5 mU per particle. In certain embodiments, the activity of the alginate lyase enzyme is between about 1.5 mU and 2.0 mU per particle. In certain embodiments, the activity of the alginate lyase enzyme is between about 2.0 mU and 2.5 mU per particle. The per particle activity of the alginate lyase enzyme can be determined as a function of the amount of alginate used to create X number of particles, and the amount of enzyme used to prepare X particles. For example, the per particle activity of the alginate lyase enzyme can be determined to be between about 0.05 mU and 2.5 mU per particle, based upon 100 mg of alginate being converted to 20,000 particles, containing between about 1 to about 50 Units of enzyme.

Furthermore, the degradation of enzyme-loaded alginate particles could also be controlled by regulating the alginate lyase enzyme activity. In order to control the catalytic degradation activity of alginate lyase, the enzyme may be complexed or pre-treated with <1 mM of Cu²⁺, Zn²⁺ and Fe³⁺ metal ions. These metal ions can inhibit enzymatic activity by approximately 90%. Other metal ions such as Mg²⁺ and Ca²⁺ at 1 mM concentration reduces the activity by 20% to 50% respectively. The free or unbound metal ions may be removed from the solution through dialysis. These metal ions can inhibit the activity of the enzyme and can be considered detrimental for the enzyme (Inoue, et al., “Functional identification of alginate lyase from the brown alga Saccharina japonica”, Sci. Rep. 2019; 9:1-11). On the contrary, the same concept is adopted in certain embodiments of the present disclosure to regulate the degradation of alginate lyase loaded alginate particles. Importantly, this enzyme shows optimum enzymatic activity at physiological temperature and pH. Thus, under in vivo condition, the enzymatic activity could be regulated solely using these metal ions to achieve the rapid (>=2 days to <=5 days) and longer (>5 days to <=30 days or >30 days) duration degrading particles.

In general, an enzyme may be immobilized into an inert or insoluble matrix. This provides resistance to physiological factors affecting the enzymatic reactions such as pH or temperature and also increase the rate of reaction. It also keeps the enzyme localized in a place (e.g, inside the particles, surface decorated, etc.). In certain embodiments of the present disclosure, immobilization/encapsulation of the modified or native alginate lyase enzyme to its sodium alginate substrate (reactive, instead of an inert matrix). Therefore, another important aspect is to avoid the initial degradation during the manufacturing of the alginate lyase loaded alginate particles from the alginate lyase-sodium alginate precursor solution. To overcome this problem following approaches are proposed for use in certain embodiments of the present disclosure.

The enzyme may be pre-treated with the metal ions inhibitors such as Cu²⁺, Zn²⁺, Fe³⁺, Mg²⁺ and Ca²⁺. These metal ions at optimum concentration without affecting the physical robustness of the particles may reduce the degradation of the particles by partially inhibiting the enzyme activity.

Another approach is to reduce the temperature of the alginate lyase-sodium alginate precursor solution from ambient to a temperature ranging from 4 to 10° C. This will reduce or cease the catalytic activity of the alginate lyase, thereby preventing the degradation of sodium alginate. In addition, the temperature of the divalent metal ions gelling bath may also be reduced to the range 4 to 10° C. This metal ion bath is used for gelling the droplets of sodium alginate-alginate lyase solution to form the divalent metal ions-complexed alginate lyase loaded sodium alginate particles.

The catalytic activity of alginate lyase may also be regulated by changing the pH of the alginate lyase-sodium alginate and gelling bath solutions. The optimum catalytic activity of this enzyme is observed at pH ranging from 6.8 to 7.5 (see, e.g., Farrés, et al., “Formation kinetics and rheology of alginate fluid gels produced by in-situ calcium release”, Food Hydrocolloids 40 (2014): 76-84). To prevent the initial degradation of sodium alginate during the preparation of alginate lyase loaded alginate particles, the pH of the alginate lyase-sodium alginate solution may be reduced to 3.0. To carry out this process, sodium acetate-acetic acid buffer, of ionic strength <1 M, preferably <0.1 M and most preferably <0.01M with a pH range 3.7-5.6. In addition, the desired pH (pH 6.5 to 3.0) of the solution may also be achieved using sodium hydroxide (>1M to <0.01M) or hydrochloric acid (>1M to <0.01M). This results in the reduction or ceasing of the alginate lyase catalytic activity. This regulation of the catalytic activity may be attributed to the unfolding of 3D conformation of alginate lyase enzyme. The ceased catalytic activity of the alginate lyase enzyme may be reversed/activated by exposing alginate lyase loaded alginate particles to the aqueous environment having pH 6.5 to 7.5 The preferred buffer to reverse the activity of the alginate lyase enzyme is phosphate buffers. The preferred ionic strength of the phosphate buffer is 0.01 M with a pH range of 6.5 to 7.5 at 20° C. The desired pH (pH 6.5 to 7.5) of the solution may also be achieved using sodium hydroxide (>1M to <0.01M) or hydrochloric acid (>1M to <0.01M). Additionally, saline or de-ionized water or an aqueous solution having a pH between 6.5-7.5 may also be used.

Therefore, a combination of the abovementioned approaches may be used efficiently to encapsulate or load the alginate lyase enzyme into the alginate particles-complexed/gelled with divalent metal ions without degrading the alginate matrix.

The precursor alginate lyase enzyme-sodium alginate solution under the appropriate conditions (low temperature and pH) needs to be gelling in a divalent metal ions bath containing one or more cryoprotectants. The composition and condition of the gelling bath are important to make desired alginate-based embolic particles. The divalent metal ion component of the gelling bath composition may be selected from the group consisting Cu²⁺, Ba²⁺, Sr²⁺, Ca²⁺, Co²⁺, Ni²⁺, Mn²⁺ and Mg²⁺ (Lee, et al., “Alginate: properties and biomedical applications,” Progress in polymer science 37, no. 1 (2012): 106-126; and Brus, et al., “Structure and dynamics of alginate gels cross-linked by polyvalent ions probed via solid state NMR spectroscopy,”Biomacromolecules 18, no. 8 (2017): 2478-2488). Divalent cation choice may also influence alginate matrix cross-linking. The binding strength of divalent metal ion with alginate is given in decreasing order Cu²⁺>Ba²⁺>Sr²⁺>Ca²⁺>Co²⁺>Ni²⁺>Mn²⁺>Mg²⁺. The preferred metal cations are Ba²⁺ and Ca²⁺. These metal ions may be used at different concentrations ranging from 0.1% w/v to 10% w/v. The addition of cryoprotectants in the gelling bath is important in two ways: (a) it helps in maintaining the sphericity and mechanical robustness of the alginate lyase loaded alginate particles during lyophilization process and (b) it also preserves the 3D conformation of the enzyme in extremely low temperatures and freezing cycles, thereby preserving the enzyme activity.

In many instances, it has been observed that the residual activity of the enzyme reduced significantly when the lyophilization of the enzymes was performed without the addition of the cryoprotectants/cryopreservation medium (Tamiya, et al., “Freeze denaturation of enzymes and its prevention with additives,” Cryobiology 22, no. 5 (1985): 446-456; and Porter, et al., “Effects of freezing on particulate enzymes of rat liver,” J. biol. Chem 205 (1953): 883-891). The cryoprotectant components may include those known in the art, such as sucrose, glycerol, ethylene glycol, sorbitol, trehalose, propylene glycol or proprietary/commercially available cryoprotectants. When these cryoprotectants are added into the gelling bath, it gets encapsulated or uniformly distributed in the matrix of sodium alginate particles (Chan, et al., “Effects of starch filler on the physical properties of lyophilized calcium-alginate beads and the viability of encapsulated cells,” Carbohydrate polymers 83, no. 1 (2011): 225-232).

Additionally, a cryoprotectant may also be used in the post-processing stage of the preparation of freeze-dried alginate lyase loaded alginate particles, instead of adding during the manufacturing process of these particles in the gelling bath containing-divalent metal ions. In this process, the droplets of the precursor alginate lyase-sodium alginate solution added into the gelling bath containing divalent metal ion only to form alginate-lyse loaded alginate particles. Following the isolation of these particles from the gelling bath, it may be soaked in a suitable cryoprotectant and subject to the freeze-drying process. Under the freeze-drying conditions, it prevents the freeze denaturation of the enzyme as well as providing the defect-free alginate lyase loaded alginate particles by preventing the collapse of the gel structure by filling the pores formed as the water is sublimed out of the matrix. The particle size may be >40 μm, <200 μm but <2000 μm. Also, this process may be used to prepare alginate-based embolic agent of different morphologies such as microfibrils, core-shell particles, Janus particles or capsules.

Furthermore, in certain embodiments, the present disclosure provides the preparation of both radiopaque and drug-loaded alginate lyase loaded alginate particles. To achieve this, a composition of divalent metal ions containing Ca²⁺ ions and one of the following x-ray contrasting metal ions such as barium, gadolinium and tantalum metal ions (Yu, et al., “Metal-based X-ray contrast media,” Chemical reviews 99, no. 9 (1999): 2353-2378) is proposed to be used in the gelling bath. Another proposed approach is the reconstitution of alginate lyase loaded alginate particles with commercially available radiopaque agents, which become temporarily absorbed into the matrix as the alginate matrix swells in the aqueous medium. The proposed method of loading the drugs/bioactive agents (anticancer and osteogenic) into alginate lyase loaded alginate particles involve the exposing these particles to the drug for 2 to 3 hours. The delivery of the drug in the body will be facilitated by the in situ degradation mechanism of alginate lyase loaded alginate particles.

In certain embodiments, enzyme loaded alginate microspheres may be stored over extended periods of time. In certain embodiments, metal ion complexed-enzyme is immobilized into its substrate. It is contempalted that the slow degradation of the matrix starts during storage condition. This degradation may be stopped by suspending the microspheres in the pH below 5.5. Apart from reducing the operating temperature below 10° C. to prevent the degradation of alginate microspheres, an alternative method is to freeze or vacuum dry these microspheres. This may stop the degradation of alginate microspheres. In certain embodiments, the dried spheres may be loaded into a specially designed syringe.

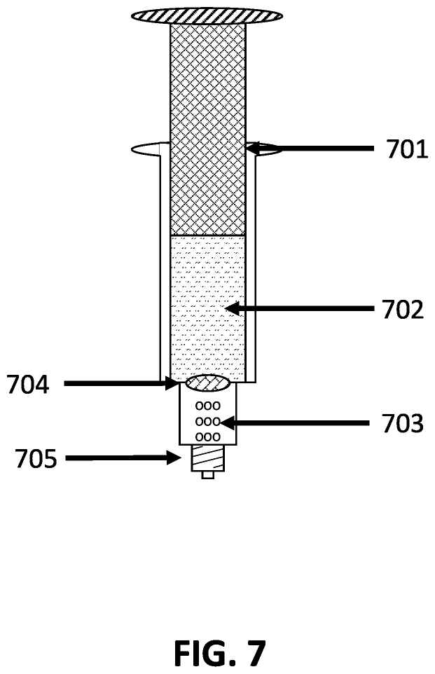

FIG. 7 illustrates a proposed design of a syringe for reconstituting and/or administering microspheres. The syringe may comprise a plunger 701, which may be in a locked or unlocked position. The syringe may also comprise a first chamber comprising a suspension medium 702, a second chamber comprising dried microspheres 703. Generally, the syringe may be constructed and arranged such that the contents of each of the chambers of the syringe are separated (e.g., fluidically) until pressure is applied to the plunger, thereby mixing the contents of each chamber (e.g., reconstituting the microspheres). It is contempalted that any multi-chamber lyophilization syringe known in the art may be used. In certain embodiments, the syringe may comprise a breakable membrane 704 separating the first chamber 701 and the second chamber 702. When pressure is applied to the plunger 701, the breakable membrane is broken and the contents of a first chamber comes into contact with the dried microspheres of a second chamber 703 to reconstitute the microspheres. In other embodiments, the syringe comprises a liquid bypass duct. In yet another embodiment, a barrier separating a first chamber and a second chamber of a syringe can comprise a one way valve. When pressure is applied to the plunger 701, the one way valve is forced open and the contents of a first chamber comes into contact with the dried microspheres of a second chamber to reconstitute the microspheres. In some embodiments, the reconstitution medium may be water for injection (WFI). Once reconstituted, the reconstituted microspheres are now ready for use. The syringe can comprise a quick connector 705 (e.g., Luer Lock connector) for connecting tubing or the like to administer the reconstituted microspheres to the subject.

Subjects

A patient treated by any of the methods or compositions described herein may be of any age and may be an adult, infant or child. In some cases, the patient is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between 2 and 20 years old, between 20 and 40 years old, or between 40 and 90 years old). The patient may be a human or non-human subject.

Any of the compositions disclosed herein may be administered to a non-human subject, such as a laboratory or farm animal. Non-limiting examples of a non-human subject include laboratory or research animals, a dog, a goat, a guinea pig, a hamster, a mouse, a pig, a non-human primate (e.g., a gorilla, an ape, an orangutan, a lemur, or a baboon), a rat, a sheep, or a cow.

Additives and Excipients

In some cases, the alginate particles or microspheres described herein may comprise an excipient that may provide long term preservation, bulk up a formulation that contains potent active ingredients, facilitate drug absorption, reduce viscosity, or enhance the solubility of the alginate particle or microsphere. An alginate particle or microsphere of the present disclosure may comprise about 1%, 2%, 3%, 4%, 5%, 6%, 7%, 8%, 9%, 10%, 15%, 20%, 25%, 30%, 35%, 40%, 45%, 50%, or greater than about 50% of the excipient by weight or by volume.

In certain embodiments, an alginate particle or microsphere of the present disclosure may comprise one or more solubilizers. As used herein, “solubilizers” include compounds such as triacetin, triethylcitrate, ethyl oleate, ethyl caprylate, sodium lauryl sulfate, sodium docusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like. An alginate particle or microsphere of the present disclosure may comprise about 1%, 2%, 3%, 4%, 5%, 6%, 7%, 8%, 9%, 10%, 15%, 20%, 25%, 30%, 35%, 40%, 45%, 50%, or greater than about 50% of the solubilizer by weight or by volume.

In some embodiments, the compositions described herein include other medicinal or pharmaceutical agents, carriers, adjuvants, such as preserving, stabilizing, wetting or emulsifying agents, solution promoters, and salts for regulating the osmotic pressure, osmolarity, and/or osmolality of the alginate particle or microsphere. In some embodiments, the compositions comprise a stabilizing agent. In some embodiments, stabilizing agent is selected from, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used.

In some embodiments, the composition comprises a suspending agent. Useful suspending agents include for example only, compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol may have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

In some embodiments, the composition comprises an additional surfactant (co-surfactant) and/or buffering agent and/or solvent. In some embodiments, the surfactant and/or buffering agent and/or solvent is a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof; c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as quaternary ammonium compounds, benzalkonium chloride, cetyltrimethylammonium bromide, and lauryldimethylbenzyl-ammonium chloride. It is contemplated that the solvent may be chosen with the intended subject in mind.

In some embodiments, the compositions disclosed herein comprise preservatives. Suitable preservatives for use in the compositions described herein include, but are not limited to benzoic acid, boric acid, p-hydroxybenzoates, phenols, chlorinated phenolic compounds, alcohols, quaternary compounds, quaternary ammonium compounds (e.g., benzalkonium chloride, cetyltrimethylammonium bromide or cetylpyridinium chloride), stabilized chlorine dioxide, mercurials (e.g., merfen or thiomersal), or mixtures thereof.

### Other Embodiments and Equivalents

The section headings used herein are for organizational purposes only and are not to be construed as limiting the subject matter described.

It is to be understood that the methods described herein are not limited to the particular methodology, protocols, subjects, and sequencing techniques described herein and as such may vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment may be combined with any other embodiment. It should be understood that numerous specific details, relationships, and methods are set forth to provide a full understanding of the features described herein. A skilled artisan, however, will readily recognize that the features described herein may be practiced without one or more of the specific details or with other methods. The features described herein are not limited by the illustrated ordering of acts or events, as some acts may occur in different orders and/or concurrently with other acts or events. Furthermore, not all illustrated acts or events are required to implement a methodology in accordance with the features described herein. Further, to the extent that the methods of the present disclosure do not rely on the particular order of steps set forth herein, the particular order of the steps should not be construed as limitation on the claims. Any claims directed to the methods of the present disclosure should not be limited to the performance of their steps in the order written, and one skilled in the art may readily appreciate that the steps may be varied and still remain within the spirit and scope of the present disclosure.

While some embodiments have been shown and described herein, it will be obvious to those skilled in the art that such embodiments are provided by way of example only. It is not intended that embodiments of the present disclosure be limited by the specific examples provided within the specification. While certain embodiments of the present disclosure have been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the embodiments of the present disclosure are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define, at least in part, the scope of the invention and that methods and structures within the scope of these claims and their equivalents be covered thereby.

It will be appreciated by those skilled in the art that changes could be made to the exemplary embodiments shown and described above without departing from the broad inventive concepts thereof. It is understood, therefore, that this disclosure is not limited to the exemplary embodiments shown and described, but it is intended to cover modifications within the spirit and scope of the present disclosure as defined by the claims. For example, specific features of the exemplary embodiments may or may not be part of the claimed invention and various features of the disclosed embodiments may be combined. The words “right”, “left”, “lower” and “upper” designate directions in the drawings to which reference is made. The words “inwardly” and “outwardly” refer to directions toward and away from, respectively, the geometric center of the fluid delivery device. Unless specifically set forth herein, the terms “a”, “an” and “the” are not limited to one element but instead should be read as meaning “at least one”.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It is to be understood that at least some of the figures and descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

### EXAMPLES

### Example 1: Alginate Lyase Enzyme Concertation Dependent Degradation of Alginate Particles

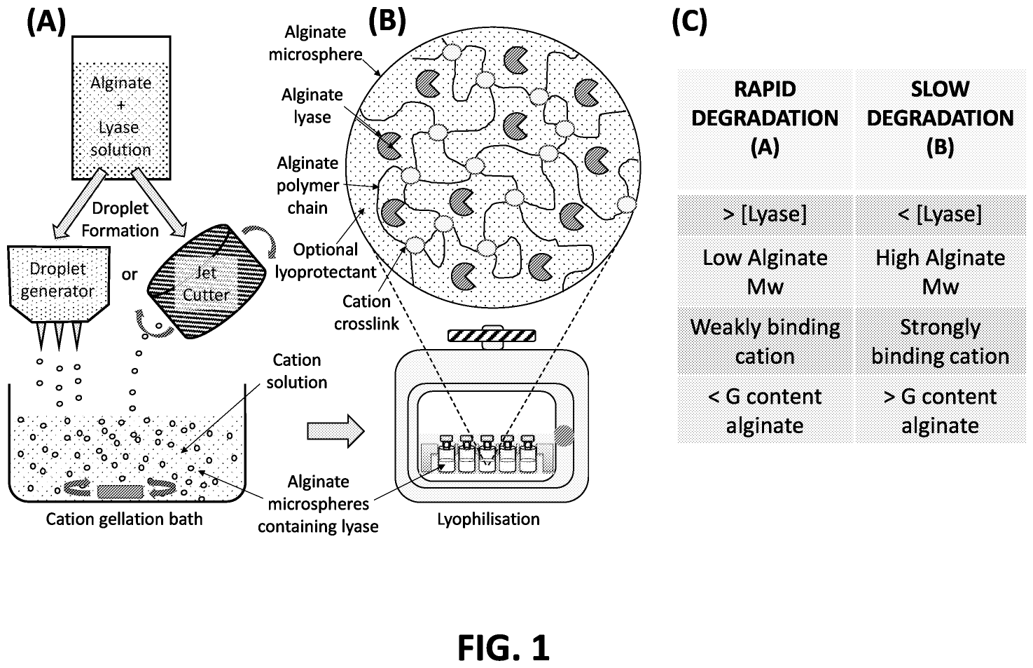

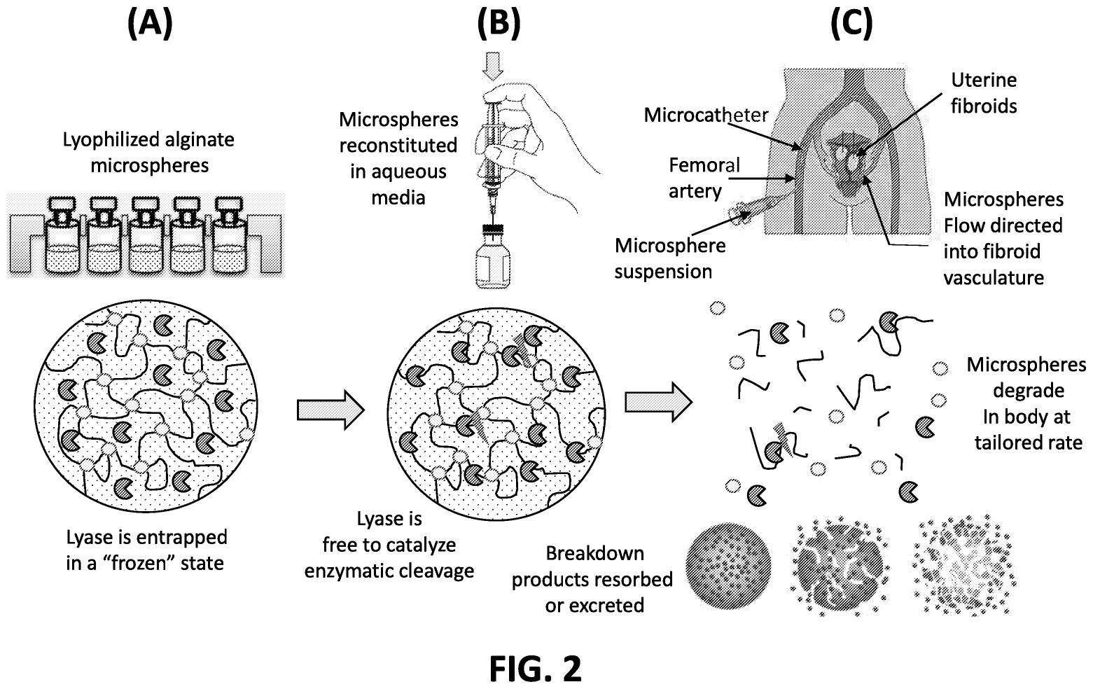

The schematic diagram for the preparation of the alginate particles is shown in FIG. 1. Sodium alginate of viscosity (5-40 cps, condition 1% w/v in water @ 25° C.) was dissolved in de-ionized water to prepare the stock solution of concentration 4% w/v. Likewise, a stock solution of alginate lyase enzyme of concentration 50 U/ml was prepared by dissolving 5 mg of enzyme powder (equivalent 50 U) in 1 ml of DI water. To prepare the alginate lyase-sodium alginate precursor solution having final concentrations of 5 U/ml or 0.5 U/ml of alginate lyase enzyme and 2% w/v of sodium alginate, 0.1 ml or 0.01 ml of alginate lyase enzyme was mixed with 0.5 ml of 4% w/v of sodium alginate and make up the volume to 1 ml with de-ionized water.

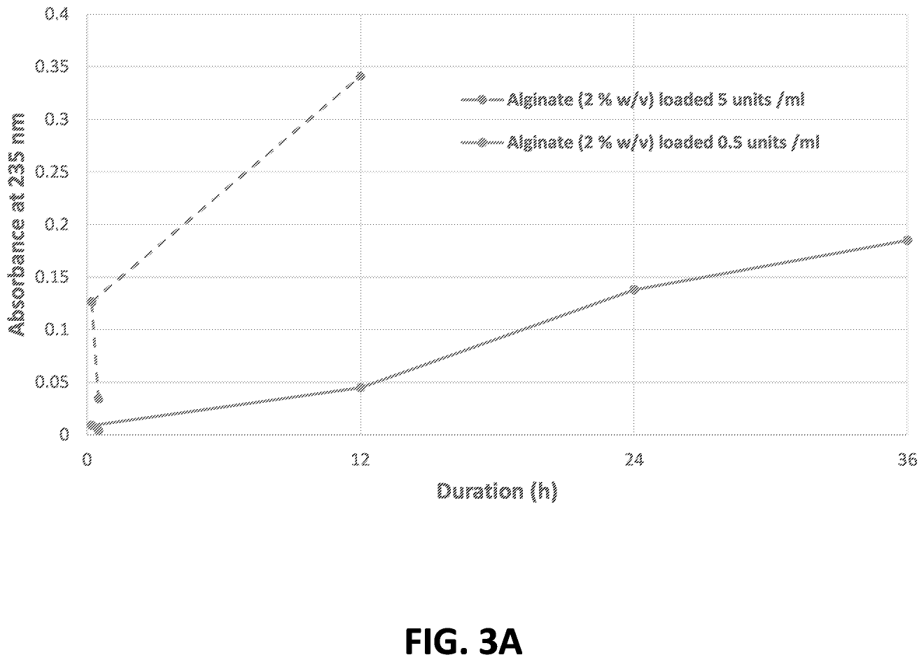

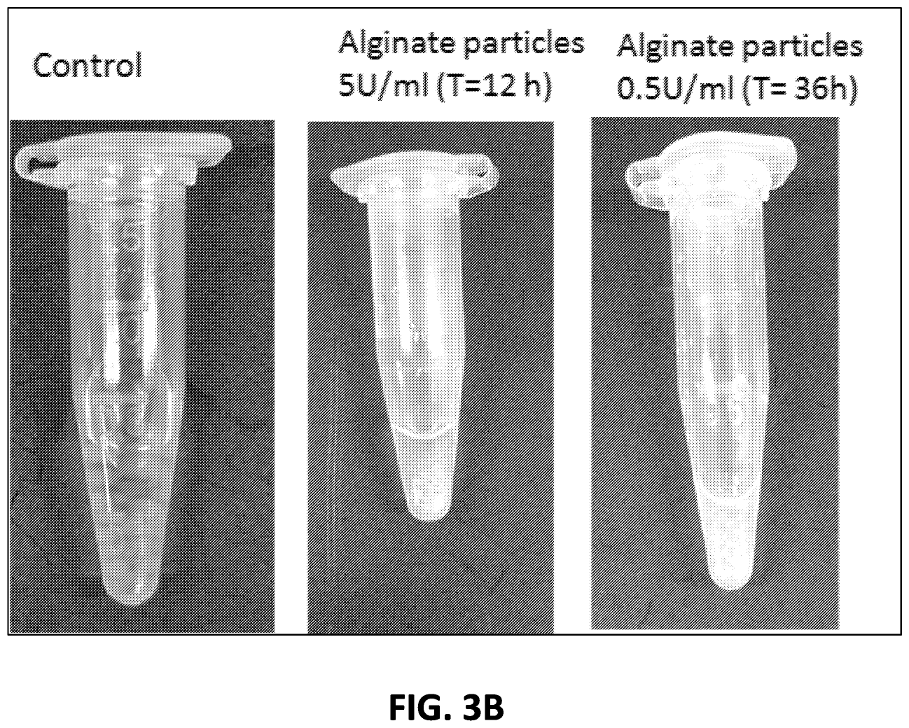

The precursor alginate lyase-alginate solution was added dropwise into the gelling bath containing 10% w/v calcium chloride under constant stirring for 5 minutes to achieve alginate lyase loaded divalent-complexed alginate particles. Then, the particles were isolated by sieving or centrifugation and washed with de-ionized water three times for 1 minute each to remove excess or calcium chloride. Washed alginate lyase loaded divalent-complexed alginate particles were dispersed in 10 mM phosphate buffer at pH 6.8 and incubated at 37° C. for the desired duration to evaluate the degradation of alginate particles. The degradation of calcium ion complexed alginate particles loaded with 5 units (U) and 0.5 U of alginate lyase enzyme shown in FIG. 3 (a). The alginate particles loaded with 5 U of enzyme rapidly degraded in 12 h, whereas the particle loaded with 0.5 U of enzyme degraded at a slower rate and unable to reach the absorbance level similar to 5 U loaded alginate particle after 36 hours. From FIG. 3(b), 5 U loaded alginate lyase loaded alginate particles were completely degraded in 12 h, whereas 0.5 U alginate lyase loaded alginate particles samples showed the partially degraded particles in 36 h. In the control sample (without enzyme), the calcium ion complexed alginate particles remained intact. These results demonstrated the alginate lyase enzyme concertation dependent degradation of alginate particles.

### Example 2: In Vitro Biocompatibility of Alginate Particles

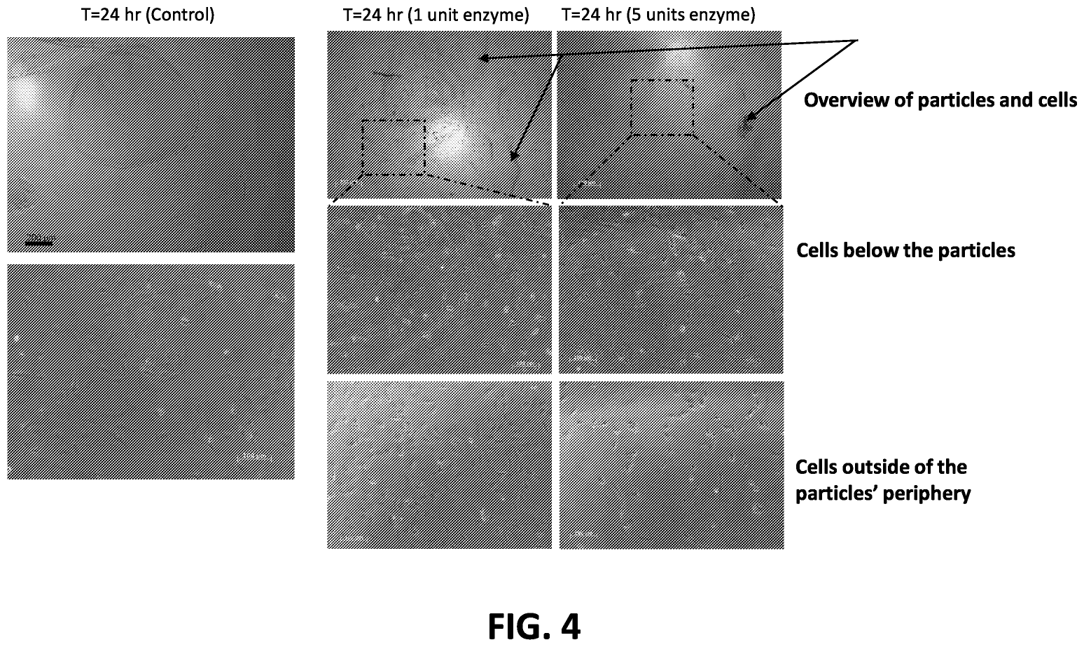

Two different calcium ion-complexed alginate particles were prepared loaded with 1 U and 5 U of alginate lyase enzyme. To evaluate the biocompatibility of particles, the morphology and viability of the cells were observed through a light microscope as shown in FIG. 4. Cells were seeded in a 24 well-plate with the cell density of 10⁴ cells per ml. Cells were cultured under 37° C., 5% CO₂ and 95% relative humidity in alpha-MEM containing 10% fetal bovine serum and 1% penicillin and streptomycin. At least 10 particles of size 2-3 mm were added in the 24 well-plate and incubated for 24 hr. In control samples, intact particles can be observed with no detrimental influence on the viability and morphology of osteoblast cells. Alginate particles loaded with 5 U of alginate lyase enzyme is completely degraded (indicated by the debris of the degraded alginate particles), whereas 1 U of alginate lyase enzyme loaded alginate particles are irregularly shaped. The cells are viable with flattened morphology below the degraded particles. This data demonstrated the in vitro biocompatibility of the alginate lyase loaded calcium-complexed alginate particles.

### Example 3: Ex Vivo Degradation of Alginate Lyase Loaded Divalent Metal Ion-Complexed Alginate Particles

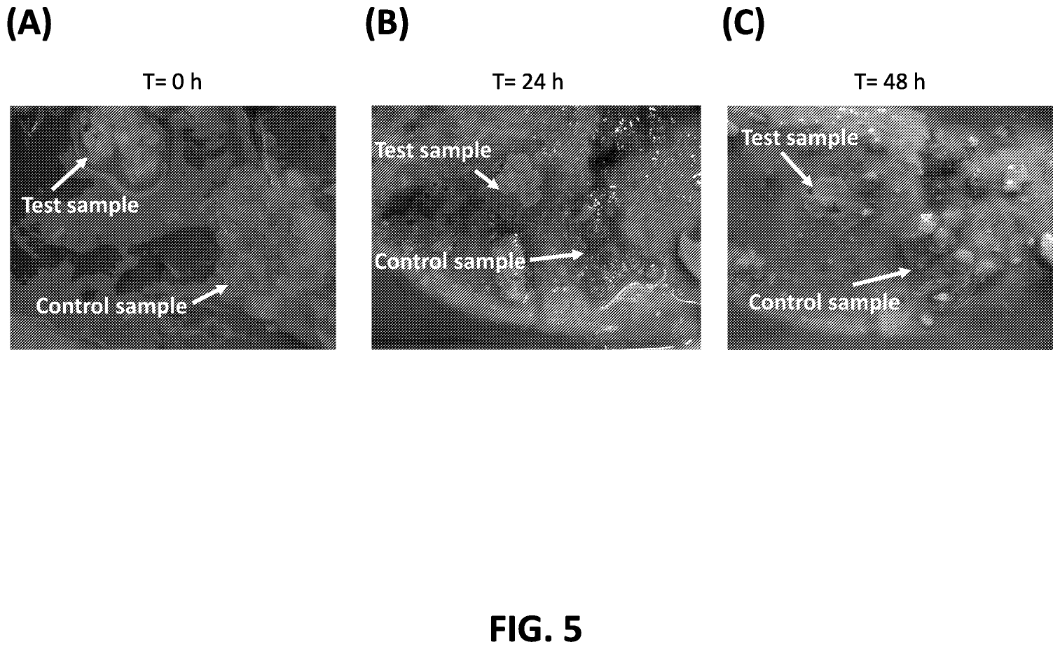

In this test, 5 U of alginate lyase enzyme was loaded into the calcium ion-complexed alginate particles and control particles (without enzyme) and placed it onto the liver (bovine) immersed in saline. To evaluate the degradation of particles, the liver was kept in an oven with a temperature set at 37±1° C. and morphological change in the particles was observed for 48 hours. From FIG. 5, the alginate lyase loaded alginate particles lost the shape in 48 hours and a film of white residue can be observed. On the other hand, the control particles maintain the shape for 48 hours. A black film on the control particles can be observed which might be the formation of biofilm.

### Example 4: pH-Dependent Regulation of Alginate Lyase Enzyme Conformation/Activity

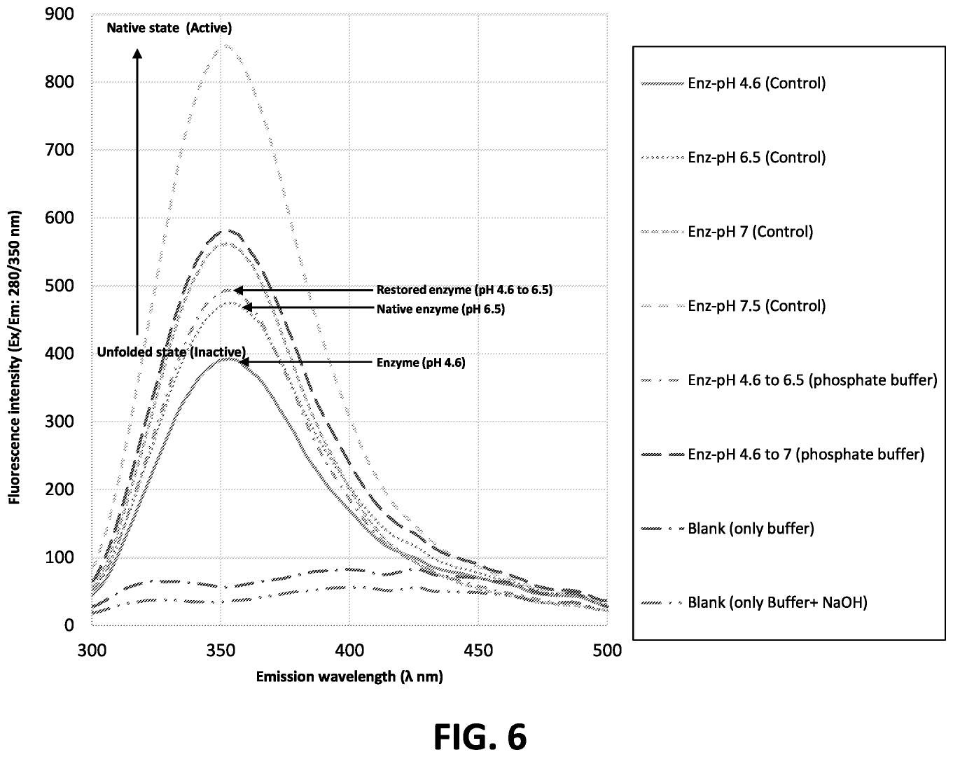

To study, the pH-dependent regulation of lyase enzyme conformation/activity, the alginate lyase enzyme was exposed to different pH and subjected to the fluorescence spectroscopy. In general, an open conformation of enzyme inactivates or reduces the enzyme catalytic activity, whereas further stabilization of the native structure improves the catalytic activity of the enzyme. From FIG. 6, the fluorescence of native enzyme (1 U/ml) in acidic pH 4.6 (acetate buffer) is at a lower level when compared to the native enzyme at pH 7.0 (10 mM, phosphate buffer). The reduction in fluorescence in acidic pH indicated the open conformation of the enzyme. When the pH of enzyme solution changed to pH 4.6 to 7.0, the fluorescence recovered or enhanced is found to be at a level similar to the native enzyme at pH 7.0. This demonstrated the reversible conformation of the alginate lyase enzyme in response to change in the pH of the solution with reactivation of enzyme activity. Therefore, by changing the pH of alginate lyase-alginate precursor or the gelling bath solutions, the initial degradation of particles during the manufacturing process of alginate lyase loaded divalent metal ion particles. On reconstituting the alginate-lyase enzyme loaded di-valent metal ions complexed alginate particles in an aqueous solution having neutral pH (6.5 to 7.5), the activity of the alginate lyase enzyme can be restored to get the tailored degradation of the alginate particles.

1. A self-degrading alginate particle, comprising:
alginate molecules having one or both of (i) a predetermined molecular weight, and (ii) a predetermined ratio of β-D-Mannuronic acid (M) blocks to α-L-Guluronic acid (G) blocks;
alginate lyase enzymes; and
metal ions to cross-link the alginate molecules.

2. The alginate particle of claim 1, wherein a degradation of the alginate particle in vivo or in vitro is controlled by one or more of the predetermined molecular weight of the alginate molecules, the predetermined ratio of M to G blocks, a concentration of the alginate lyase enzyme, a concentration of the metal ions, and a binding affinity of the metal ions.

3. The alginate particle of claim 1, wherein at least one of (i)-(x) applies:
(i) the predetermined molecular weight of the alginate molecules is greater than about 100 kilodaltons (kD), greater than about 200 kD, or greater than about 800 kD,
(ii) the predetermined ratio of M to G blocks is about 50:50, about 55:45, about 60:40, about 65:35, about 70:30, about 75:25, about 80:20, about 85:15, about 90:10, or about 95:5,
(iii) the predetermined ratio of M to G blocks is about 50:50, about 45:55, about 40:60, about 35:65, about 30:70, about 25:75, about 20:80, about 15:85, about 10:90, or about 5:95,
(iv) the concentration of the alginate lyase enzyme is between 0.05 mU (milliunits) and about 2.5 mU per particle,
(v) the concentration of the alginate lyase enzyme is between about 0.05 nU (nanounits) and about 0.05 mU per particle,
(vi) the metal ion is a cation,
(vii) the activity of the alginate lyase enzyme is less than about 0.05 nU per particle,
(viii) a diameter of the alginate particle is between about 40 microns (μm) and about 2000 μm, between about 40 μm and about 1000 μm, or between about 40 μm and about 200 μm,
(ix) a sphericity of the alginate particle is at least about 0.7, at least about 0.75, at least about 0.8, at least about 0.85, at least about 0.9, at least about 0.95, or at least about 0.99, or
(x) the alginate molecules comprise oxidized alginate molecules.

4.-8. (canceled)

9. The alginate particle of claim 3, wherein at least one of (a)-(e) applies:
(a) the alginate particle of (ii) degrades over a period of less than about 5 days or over a period of greater than about 2 days,
(b) the alginate particle of (iii) degrades over a period of between about 5 days and about 30 days,
(c) the alginate particle of (iv) degrades over a period of less than about 5 days,
(d) the alginate particle of (v) degrades over a period of between about 5 days and about 30 days, or
(e) the alginate particle of (vii) degrades over a period greater than about 30 days.

10.-21. (canceled)

22. The alginate particle of claim 1, wherein at least one of (i)-(iv) applies:
(i) the metal ion is a cation selected from the group consisting of Cu²⁺, Ba²⁺, Sr²⁺, Ca²⁺, Co²⁺, Ni²⁺, Mn²⁺ and Mg²⁺,
(ii) the alginate particle further comprises one or more alginate lyase inhibitors independently selected from the group consisting of Cu²⁺, Zn²⁺, Ca²⁺ and Mg²⁺,
(iii) the alginate particle further comprises a cryoprotectant,
(iv) the alginate particle further comprises a therapeutically effective amount of an active ingredient.

23.-29. (canceled)

30. The alginate particle of claim 22, wherein the cryoprotectant of (iii) is selected from the group consisting of sucrose, glycerol, ethylene glycol, sorbitol, trehalose, and propylene glycol.

31.-33. (canceled)

34. A method of inducing a self-degrading embolism in a subject in need thereof, comprising administering a plurality of the alginate particles of claim 1 into a blood vessel of the subject.

35. The method of claim 34, wherein the blood vessel is a geniculate artery.

36-69. (canceled)

70. A method of preparing a self-degrading alginate particle, the method comprising:
obtaining a first composition comprising alginate microspheres, said alginate microspheres comprising alginate molecules having one or both of (i) a predetermined molecular weight, and (ii) a predetermined ratio of β-D-Mannuronic acid (M) blocks to α-L-Guluronic acid (G) blocks;
mixing the first composition with a second composition comprising alginate lyase enzymes and metal ions, thereby creating a mixture; and
preparing a self-degrading alginate particle from the mixture.

71. The method of claim 70, further comprising,
inhibiting degradation of the alginate molecules in the mixture by one or both of:
(i) maintaining a pH of the mixture at less than about 6.5; and
(ii) maintaining a temperate of the mixture at less than about 10 degrees Celsius (° C.).

72. The method of claim 71, wherein the pH of the mixture is maintained at between about 3 and about 6.5 and/or the temperature of the mixture is maintained at between about 4° C. and about 10° C.

73. (canceled)

74. The method of claim 70, wherein the preparing comprises performing a water-in-oil emulsion technique or a droplet technique.

75. The method of claim 70, further comprising:
reconstituting the self-degrading alginate particle in a solution having a pH of between about 6.8 and about 7.5, and/or
adding a cryoprotectant to the alginate particle prior to lyophilizing the alginate particle.

76. The method of claim 70, wherein a degradation of the alginate particle in vivo or in vitro is controlled by one or more of the predetermined molecular weight of the alginate molecules, the predetermined ratio of M to G blocks, a concentration of the alginate lyase enzyme, a concentration of the metal ions, and a binding affinity of the metal ions.

77. (canceled)

78. The method of claim 70, wherein at least one of (i)-(x) applies:
(i) the predetermined molecular weight of the alginate molecules is greater than about 100 kilodaltons (kD), greater than about 200 kD, or greater than about 800 kD,
(ii) the predetermined ratio of M to G blocks is about 50:50, about 55:45, about 60:40, about 65:35, about 70:30, about 75:25, about 80:20, about 85:15, about 90:10, or about 95:5,
(iii) the predetermined ratio of M to G blocks is about 50:50, about 45:55, about 40:60, about 35:65, about 30:70, about 25:75, about 20:80, about 15:85, about 10:90, or about 5:95,
(iv) the activity of the alginate lyase enzyme is between 0.05 mU (milliunits) and 2.5 mU per particle,
(v) the activity of the alginate lyase enzyme is less than about 0.05 nU (nanounits) per particle,
(vi) the concentration of the alginate lyase enzyme is between about 0.05 nU to 0.05 mU per particle,
(vii) the metal ion is a cation,
(viii) a diameter of the alginate particle is between about 100 microns (μm) and about 2000 μm, between about 100 μm and about 1000 μm, or between about 100 μm and about 200 μm.
(ix) a sphericity of the alginate particle is at least about 0.7, at least about 0.75, at least about 0.8, at least about 0.85, at least about 0.9, at least about 0.95, or at least about 0.99, or
(x) the alginate molecules comprise oxidized alginate molecules.

79.-82. (canceled)

83. The method of claim 78, wherein at least one of (a)-(e) applies:
(a) the alginate particle of (ii) degrades over a period of less than about 5 days or over a period of greater than about 2 days,
(b) the alginate particle of (iii) degrades over a period of between about 5 days and about 30 days,
(c) the alginate particle of (iv) degrades over a period of less than about 5 days,
(d) the alginate particle of (vi) degrades over a period of between about 5 days and about 30 days, or
(e) the alginate particle of (v) degrades over a period of between about 5 days and about 30 days.

84.-95. (canceled)

96. The method of claim 70, wherein at least one of (i)-(iv) applies:
(i) the metal ion is a cation selected from the group consisting of Cu²⁺, Ba²⁺, Sr²⁺, Ca²⁺, Co²⁺, Ni²⁺, Mn²⁺, and Mg²⁺,
(ii) the alginate particle further comprises one or more alginate lyase inhibitors independently selected from the group consisting of Cu²⁺, Zn²⁺, Fe³⁺, Ca²⁺ and Mg²⁺,
(iii) the alginate particle further comprises a cryoprotectant,
(iv) the self-degrading alginate particle comprises a therapeutically effective amount of an active ingredient.

97.-104. (canceled)

105. The method claim 96, wherein the cryoprotectant of (iii) is selected from the group consisting of sucrose, glycerol, ethylene glycol, sorbitol, trehalose, and propylene glycol.

106.-112. (canceled)

113. The alginate particle of claim 1, wherein the alginate lyase enzymes are entrapped by the cross-linked alginate molecules.

114.-116. (canceled)

117. The method of claim 70, wherein subsequent to mixing the first composition with the second composition, the metal ions cross-link the alginate molecules, and the alginate lyase enzymes are entrapped by the cross-linked alginate molecules.
